(19) United States
(12) Patent Application Publication
(10) Pub. No.: US 2002/0012912 A1
(43) Pub. Date: Jan. 31, 2002

(54) PARALLEL COMBINATORIAL LIBRARIES FOR CHIRAL SELECTORS

(76) Inventors: TINGYU LI, NASHVILLE, TN (US); THOMAS M. HARRIS, NASHVILLE, TN (US); YAN WANG, NASHVILLE, TN (US)

Correspondence Address: RICHARD S. MYERS, JR., 414 UNION STREET, SUITE 2020, BANK OF AMERICA PLAZA, NASHVILLE, TN, 37219, US

(21) Appl. No.: 09/318,186

(22) Filed: May 25, 1999

(51) Int. Cl. C12Q 1/68; G01N 33/53; G01N 33/567; C12P 19/34; C07K 1/04

(52) U.S. Cl. 435/6; 435/7.1; 435/7.2; 435/91.1; 436/501; 436/518; 530/333; 530/334

(57) ABSTRACT

A method to screen parallel combinatorial libraries for chiral selectors, for instance, a parallel library screening procedure demonstrating the chiral resolution of racemic analyte such as racemic (1-naphthyl)leucine ester. The method involves synthesis of potential chiral selectors on polymeric synthesis resins and the rapid screening of selectors directly on the resins with batch incubation, followed by circular dichroism measurement. The method does not require pre-immobilization of the analyte. The identified chiral selector is then attached onto a support and employed to resolve the racemic analyte into its R-enantiomer and S-enantiomer.

### TECHNICAL FIELD

The present invention relates, in general, to preparation of combinatorial libraries and screening of chiral selectors. More particularly, the present invention relates to preparation of parallel combinatorial libraries for the screening of chiral selectors, without the necessity for pre-immobilization of the analyte. The invention is useful for the resolution of a racemic mixture to determine which enantiomer of the racemic mixture has better activity, selectivity, or other drug requirements.

### Table of Abbreviations

Table of Abbreviations

| | |
|---|---|
| Å | angstrom |
| Abu | 4-aminobutyric acid |
| Ala | alanine |
| AmPS resin | amino methylated polystyrene resin |
| | (a resin with a hydrophobic surface) |
| AmTG resin | amino methylated polystyrene resin |
| | (another polystyrene resin, but with a |
| | hydrophilic surface) sold under the |
| | trade name TENTAGEL or the |
| | trademark NOVASYN ® TG |
| Anth | 9-anthroyl |
| Bz | benzoyl |
| CD | circular dichroism |
| DCM | dichloromethane |
| DIPEA | N,N-diisopropylethylamine |
| DMF | N,N-dimethylformamide |
| Dnb | 3,5-dinitrobenzoyl |
| Fmoc | N-(9-fluorenylmethoxycarbonyl) |
| Gly | glycine |
| HPLC | high performance liquid |
| | chromatography |
| Hex | hexane |
| OH | hydroxyl |
| IPA | isopropanol |
| L | levo (left-rotating) |
| Leu | leucine |
| μm | micrometer |
| mg | milligram |
| ml | milliliter |
| mmol | millimole |
| min. | minute |
| Naph | 2-naphthoyl |
| nm | nanometer |
| PAA | polyacrylamide |
| Ph | phenyl |
| \mathring{P} | polymer |
| Pro | proline |
| PyBOP | benzotriazolyloxy-tris |
| | [pyrrolidino]-phosphonium |
| | hexafluorophosphate |
| TLC | thin layer chromatography |
| UV | ultraviolet |

### Background Art

A method to screen mixture combinatorial libraries for chiral selectors based on the reciprocity of chromatographic separation and the application of enantiomeric libraries has been reported. See, Wu, Wang, Yang, and Li, “Screening of Mixture Combinatorial Libraries for Chiral Selectors: A Reciprocal Chromatographic Approach Using Enantiomeric Libraries”, Vol., 71, Anal. Chem., pp. 1688-1691 (1999). While a large number of compounds can be synthesized and screened efficiently in this mixture library approach, analytes of interest need to be immobilized prior to the reciprocal chromatographic screening.

In contrast to a mixture library approach, library components are synthesized and screened individually in a parallel library approach, as described by Chaiken and Janda, “Molecular Diversity and Combinatorial Chemistry”, American Chemical Society Conference, Washington, D.C. (1996). The parallel library approach is similar to a traditional chemistry method in which a compound in its pure form is synthesized and evaluated.

However, the success of a parallel library approach depends highly on the efficiency of the synthesis and screening of the library components. There have been two reports on the application of parallel libraries to the development of screening for chiral selectors.

One report is a reciprocal chromatographic assay of racemic library components. More specifically, Lewandowski, Murer, Svec, and Frechet, “Highly Selective Chiral Recognition on Polymer Supports: Preparation of a Combinatorial Library of Dihydropyrimidines and Its Screening for Novel Chiral HPLC Ligands”, Chem. Communications (1998) describe that one enantiomer of a racemic analyte was immobilized onto a chromatographic support, namely poly[(N-methyl)aminoethyl methacrylate-co-methyl methacrylate-co-ethylene dimethacrylate] beads, and that the resolution of individual racemic library components was tested using this analyte stationary phase. Unfortunately, this parallel library screening procedure, like the above-described mixture library screening procedure, requires immobilization of the analyte. The other report is a conference poster presentation by employees from Regis Technologies, Inc., Morton Grove, Ill. (U.S.A.). More specifically, Protopopova, Bhat, and Weich, “Microscale Synthesis and Screening of Chiral Stationary Phases”, Poster Presentation at HPLC Conference, St. Louis, Mo. (May 2-8, 1998) describe synthesis of parallel libraries of potential chiral selectors on silica gel but fail to disclose details of their screening procedure. Moreover, one of the inventors of the instant case has found through his own research that reactions on silica gel are often rather complicated, and their efforts to synthesize peptides onto silica gel have been discouraging, and therefore, the silica gel is unsuitable for the preparation of a parallel library. (See below discussion, vis-à-vis coupling of Abu and Leu to silica gel.)

Therefore, a more efficient screening method is desirable in order to develop chiral selectors from parallel libraries.

### SUMMARY AND OBJECTS OF THE INVENTION

Accordingly, the present invention provides a method for screening chiral selectors from a parallel library. The method comprises forming a parallel library by individually synthesizing potential chiral selectors onto a polymeric synthesis resin. All the selectors possess either a levo rotational orientation or a dextro rotational orientation, and the selectors are not a mixture of both levo rotational orientation and dextro rotational orientation. Next, each individual chiral selector, attached on the resin, is incubated with an analyte having a mixture of a R-enantiomer and a S-enantiomer. Then, the resultant from the incubation is analyzed to identify which chiral selector selectively adsorbed one of the R-enantiomer and the S-enantiomer. The method may further include attaching the identified chiral selector onto a support and resolving the racemic analyte into the R-enantiomer and the S-enantiomer with the attached chiral selector on the support. The method may further include at least one selector that may be a negative control.

Hence, it is an object of the invention to provide a screening method that does not require first immobilizing the analyte in order to screen for potential chiral selectors.

Some of the objects of the invention having been stated above, other objects will become evident as the description proceeds, when taken in connection with the accompanying Figures and Laboratory Examples as best described below.

### BRIEF DESCRIPTION OF THE FIGURES

FIG. 1 is a graph showing the circular dichroism spectra of R- and S-(1-naphthyl)leucine ester with a solute concentration of 2 mg/ml and a cell length of 1 mm.

FIG. 2 is a chromatogram showing resolution of racemic (1-naphthyl)leucine ester using an immobilized Dnb-Leu silica gel stationary phase, with column size: 50×4.6 mm. mobile phase: 20% IPA in hexanes, flow rate: 1.2 ml/minute, UV detector (254 nm), and the dead time tₒ=0.48 minutes.

### DETAILED DESCRIPTION OF THE INVENTION

The synthesis of potential chiral selectors on polymeric synthesis resins and the screening of these selectors on the resin using a batch incubation procedure has been found to be more rapid, as well as much more reliable, than the above-noted synthesis on silica gel. The screening procedure developed has been found to be a convenient batch incubation assay which may be based on the measurement of circular dichroism that indentifies which chiral selector is desirable out of the batch of selectors, without the need for packing any columns in order to do the indentification.

In this procedure, potential chiral selectors were first synthesized individually onto a solid phase polymeric synthesis resin. An analyte (containing a mixture of a R-enantiomer and a S-enantiomer) in an appropriate solvent was then allowed to incubate with each individual library component on the resin. The analyte mixture could be racemic, where the R-enantiomer and the S-enantiomer are in a 1:1 ratio, but this is not necessary, and they may be in any ratio in the mixture.

Then, the enantiomeric ratio of the analyte in the supernatant was analyzed to determine if selective adsorption of one of the two enantiomers to the resin was evident. Once such selective adsorption had been identified, the chiral selector was attached to a chromatographic support, and chiral resolution of the racemic analyte was evaluated.

To demonstrate the general principle, studied was the chiral resolution of the following S- and R-enantiomers of racemic (1-naphthyl) leucine ester

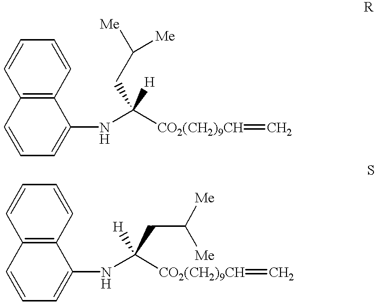

Synthesis of (1-naphthyl)leucine ester and separation of the enantiomers is described in Pirkle and Pochapsky, “A New, Easily Accessible Reciprocal Chiral Stationary Phase for the Chromatographic Separation of Enantiomers”, Vol. 108, J. Am. Chem. Soc., No. 2, pp. 183-187 (1986), and Pirkle, Deming, and Burke, “A Chiral Stationary Phase Which Affords Unusually High Levels of Enantioselectivity”, Vol. 3, Chirality, pp. 183-187 (1991).

As can be seen from the above Table of Abbreviations, each of Dnb, Naph, Bz, and Anth is a moiety that contains carbonyl (namely, C═O), and as can be seen from the below Example II, each becomes a carboxylic acid (namely, an acid with COOH). As is well known, peptides are amides formed from the interaction of amino moieties with carboxylic acid moieties. Hence, in the following description of a preferred embodiment, peptides are synthesized onto a polymeric resin.

More particularly, chosen for this purpose was a small parallel L-(4×4) peptide library, comprising [Module 1]-[Module 2]-Abu-AmPS, where the building blocks for Module 1 are individually each of Dnb, Naph, Bz, and Anth, and the building blocks for Module 2 are individually each of the amino acids, L-Leu, L-Ala, Gly, and L-Pro, and the polymeric resin is AmPS. The Gly in Module 2 served as a negative internal standard.

Each individual library component (i.e., potential chiral selector) was synthesized onto AmPS resin, a derivative of the widely used Merrifield resin, using a HI-TOP filter plate manual synthesizer. In this synthesizer, an aliquot of resin can be added to each of the wells of a 96-well filter plate. By adding individual amino acids into each well containing resin, up to 96 peptides can be synthesized in one run. This HI-TOP system allows for quick filtration between steps of the procedure without the need to transfer resins out of the plate that holds the wells, and the overall synthetic efficiency is high.

In terms of the chemistry utilized, Fmoc chemistry was chosen to synthesize this peptide library and a representative schematic of the chemistry involved, for the attachment of the Dnb-Leu member of the parallel library to the Abu-AmPS resin as the example, is illustrated as follows:

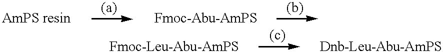

where (a) is Fmoc-Abu, PyBop; (b) is (1) piperidine, (2) Fmoc-Leu-OH, PyBop; (c) is (1) piperidine, (2) Dnb-OH, PyBop; and AmPS resin is NH₂—CH₂—Ph-\hat{P}. Other library members were synthesized following essentially similar reactions.

Using AmTG resin, another popular peptide polystyrene synthesis resin (which, in contrast to AmPS resin whose surface is hydrophobic, has a hydrophilic surface as it is coated with a layer of polyethylene glycol), the library on AmTG resin was synthesized following procedures essentially similar to those employed for the synthesis on AmPS resin.

Via circular dichroism, the ellipticities were measured for each individual component of both the library using AmPS and the library using AmTG.

As can be seen from the data in the Table below in Example IV, the same two potential chiral selectors (Dnb-Ala and Dnb-Leu) were identified from the measurements on AmTG resin as with the measurements on AmPS resin. The magnitude of the ellipticities measured with the same selector concentration, however, was smaller with the AmTG resin when compared with the values on the AmPS resin.

Although these results indicate that the kind of polymeric resin does influence the screening outcome, resins other than AmPS resin and AmTG resin are useful, as long as large separation factors are being sought. It is contemplated that also useful are polyacrylamide resins (PAA resins), such as polyacrylamide cross-linked with an alkyl diamine or cross-linked with bisacrylamide sold under the trade name SPAR-50 by Advanced ChemTech (Louisville, Ky., U.S.A.) and the trade name PEGA by Novabiochem (San Diego, Calif., U.S.A.), in addition to use of polystyrene resins (PS resins). With significant chiral separation, chiral selectivity observed in the batch process on one polymeric synthesis resin is very likely to be observed on another polymeric synthesis resin, although the magnitude may be different.

Polymers useful as synthesis resins are generally cross-linked, lending themselves well to forming rigid structures, as opposed to polymers like polyethylene that are characterized in the art as flexible since they lend themselves well to forming flexible films. All synthesis resins should work well in the present invention.

Hence, the peptide based parallel library was synthesized in high efficiency on both the AmPS and the AmTG resins. For example, the coupling yields of Fmoc-Abu-OH to both resins were close to 100%. Specifically, found was 110% for AmPS resin and 92% for AmTG resin, as determined respectively by calculations based upon the manufacturer's certification, and by the Fmoc cleavage reaction, according to NovaBiochem Catalog & Peptide Synthesis Handbook, 1999, page S43, “Method 12: Estimation of Level of First Residue”, which should be a very reliable measurement of the extent of the coupling reaction of the amino acid to the resin.

The calculated yield of 110%, based upon manufacturer's certification of the AmPS resin, indicated that the actual surface amino concentration was higher than the manufacturer's suggested value. The surface Abu concentration was determined to be 0.44 mmol/g based on the Fmoc cleavage method.

Moreover, the subsequent coupling of Fmoc-Leu to Abu-AmPS, in which the Abu amount was determined accurately by the Fmoc cleavage method, showed that couplings of Fmoc-Leu-OH to Abu-AmPS and Abu-AmTG were achieved with 98% coupling yields in both cases.

In contrast, coupling of Fmoc-Abu-OH to aminopropyl silica gel (ALLSPHERE, sold by Alltech of Deerfield, Ill., U.S.A.) was achieved in only 64% yield. With a 64% coupling yield, a significant amount of the aminopropyl group still remained on the silica gel after the coupling reaction. Furthermore, coupling of Fmoc-Leu-OH to Abu-silica gel could not be carried out in high yield either (yield still<83%). Therefore, a complicated mixture rather than the intended pure library component will most likely be formed in the solid phase synthesis of the individual library member on silica gel.

Demonstrated was that the rapid screening of chiral selectors from a parallel library is feasible. Keys to the success of this approach are the efficient synthesis of potential chiral selectors onto polymeric synthesis resins (for instance, AmPS and AmTG) and the rapid screening of the potential selectors with circular dichroism measurement. This approach does not require immobilization of the racemic analyte.

### Laboratory Examples

General Supplies And Equipment

Solid phase polymeric synthesis resins (AmPS and AmTG) and amino acid derivatives were purchased from NovaBiochem (San Diego, Calif., U.S.A.). AmPS and AmTG are also available from Advanced ChemTech (Louisville, Ky., U.S.A.) These polystyrene resins are generally cross-linked with divinylbenzene. All other chemicals and solvents were purchased from either Aldrich (Milwaukee, Wis., U.S.A.), Fluka (Ronkonkoma, N.Y., U.S.A.), or Fisher Scientific (Pittsburgh, Pa., U.S.A.).

HPLC grade silica gel (particle size 5 μm, pore size 80 Å, and surface area 220 m²/g) sold under the trade name ALLSPHERE was purchased from Alltech (Deerfield, Ill., U.S.A.). Silica gel (32-63 μm) sold under the trade name SELECTO and purchased from Fisher Scientific (Pittsburgh, Pa., U.S.A.) was used for flash column chromatographic purification of target compounds.

Thin layer chromatography was completed using EM silica gel 60 F-254 TLC plates (0.25 mm) manufactured by Merck (Darmstadt, Germany) and sold in the United States of America by Aldrich (Milwaukee, Wis., U.S.A.). HPLC analyses were completed with an analytical gradient system sold under the trade name System Gold from Beckman (Fullerton, Calif., U.S.A.).

Circular dichroism was measured with a J-720 spectropolarimeter from JASCO (Eaton, Md., U.S.A.), while UV spectrum was measured with a UV 201 spectrometer from Shimadzu (Norcross, Ga., U.S.A.). The manual synthesizer used for parallel library synthesis was sold under the trade name HI-TOP from Whatman Polyfiltronic (Rockland, Md., USA).

Elemental analyses were conducted by Atlantic Microlab, Inc. (Norcross, Ga., USA).

Determination of the amount of Fmoc group on the resins was conducted according to NovaBiochem Catalog & Peptide Synthesis Handbook, 1999, S43, “Method 12: Estimation of Level of First Residue”, and in general was performed as follows for measurement of the extent of the coupling reaction of the amino acid to the resin. About 20 mg of the resin, on which the amount of Fmoc group was to be determined, was added to 3 ml of 20% piperidine in DMF in a quartz UV cuvette. After the mixture was gently agitated for 3-5 minutes, the resin was allowed to settle to the bottom of the cuvette. The cuvette was then placed into the UV spectrophotometer and the absorbance of the sample at 290 nm was recorded with a solution of 20% piperidine in DMF in the reference cell. The amount of the Fmoc group on the resin was then determined by comparing the UV absorbance with a calibration curve generated by cleaving known amounts of Fmoc-Gly-OH.

### Example I

Preparation of Abu-AmPS resin

A mixture of Fmoc-Abu-OH (390 mg, 1.20 mmol), PyBop (625 mg, 1.20 mmol), and DIPEA (155 mg, 1.20 mmol) in DMF (10 ml) was added to 1 g (surface amino concentration, 0.40 mmol/g) of AmPS resin pre-swelled in DCM (10 minutes). After agitation at room temperature for 2 hours, the resin (Fmoc-Abu-AmPS) was collected and washed with DMF, DCM, IPA and DCM (10 ml×2).

The Fmoc protecting group was then removed by treatment of the resin with 10 ml of 20% piperidine in DMF for 20 minutes. The deprotected resin (Abu-AmPS) was collected by filtration and washed with DMF, DCM, IPA, and DCM (10 ml×2).

The calculated yield of 110% for coupling of Fmoc-Abu-OH to AmPS resin was based upon the manufacturer's certification of the AmPS resin, and indicated that the actual surface amino concentration was higher than the manufacturer's suggested value. Accordingly, the surface Abu concentration was determined using the Fmoc cleavage method and was found to be 0.44 mmol/g.

### Example II

Preparation Of Parallel L-(4×4) Library:

L-[Anth, Bz, Dnb, or Naph]—[Ala, Gly, Leu, or Pro]—Abu-AmPS

The library was synthesized using the polyfiltronic HI-TOP manual synthesizer. Specifically, 960 mg of Abu-AmPS resin, prepared as in Example I, was equally distributed with a 60 mg aliquot into each of 16 wells of a HI-TOP 96-well filter plate (0.026 mmol of Abu in each well). Then, 4 identical mixtures of L-Fmoc-Ala-OH (16.4 mg, 0.0528 mmol), PyBOP (28.0 mg, 0.0528 mmol), and DIPEA (7.0 mg, 0.053 mmol) in 0.50 ml of DMF were added to each well of a group of 4 of the 16 wells, while identical mixtures of each of the 3 respective L-Fmoc-Leu-OH (18.6 mg, 0.0528 mmol), Fmoc-Gly-OH(15.7 mg, 0.0528 mmol), L-Fmoc-Pro-OH (17.8 mg, 0.0528 mmol), and the corresponding reagents in 0.50 ml of DMF were respectively added to the other 12 wells (3 groups of 4 wells each).

After agitation for 2 hours, the resins were filtered and washed with DMF, DCM, IPA and DCM.

The Fmoc protecting group was then removed by treatment with 0.60 ml of 20% piperidine in DMF for 20 minutes, followed by washing with DMF, DCM, IPA and DCM. Each of the carboxylic acids (Anth-OH, Bz-OH, Dnb-OH and Naph-OH) was then coupled to the deprotected α-amino acid-Abu-AmPS resin with the combination necessary to produce the L-(4×4) library, L-[Anth, Bz, Dnb, or Naph]—[Ala, Gly, Leu, or Pro]—Abu-AmPS, following the procedures described above for the attachment of Dnb-Leu to the Abu-AmPS resin.

### Example III

Synthesis of the L-(4×4) library on the AmTG resin

The parallel L-(4×4) library on AmTG resin was prepared by following procedures described above in Examples I and II for the preparation of the L-(4×4) library on AmPS resin. The only difference was that the amount of the AmTG resin used was increased to 100 mg (from 60 mg used for AmPS resin) in each well, as the surface amino group concentration (0.25 mmol/g) of the AmTG resin was lower than that of the corresponding AmPS resin.

### Example IV

Screening of the parallel L-(4×4) library with circular dichroism measurement

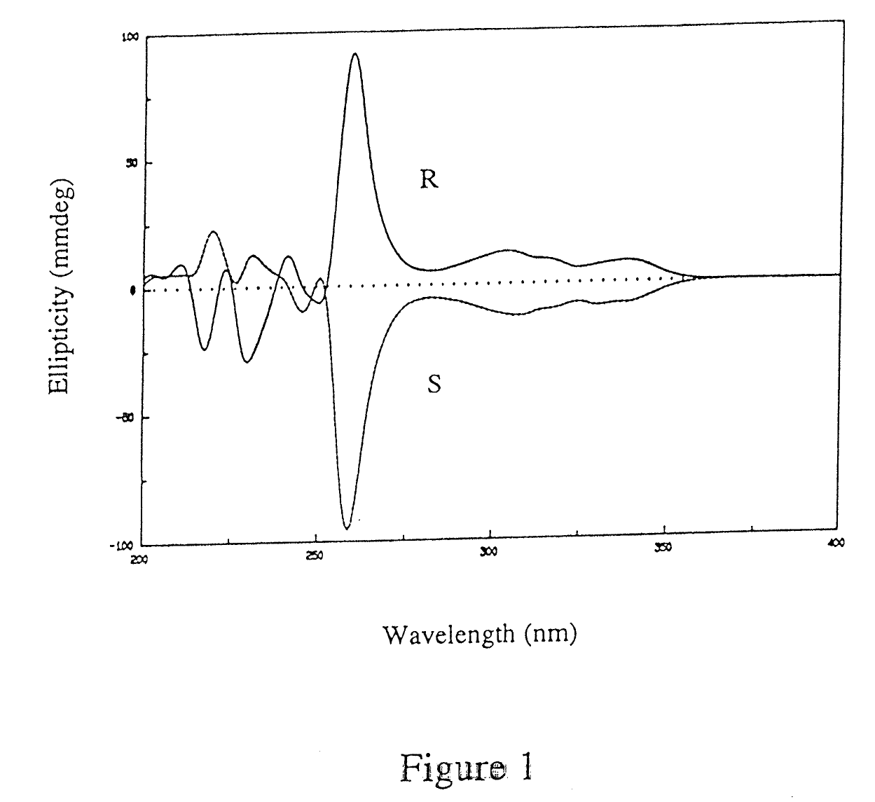

The resins, each containing 0.026 mmol of attached potential selector, from Examples II and III were transferred to 16 wells of a regular 96-well plate. To each well was added racemic (1-naphthyl) leucine ester (1.2 mg, 0.0030 mmol) in a solvent mixture of IPA:Hex (2:8, 0.6 ml). After incubation for 24 hours at room temperature (about 72° F., 22° C.), the supernatant in each well was transferred into the sample cell (volume 0.40 ml) of a JASCO J-720 CD spectropolarimeter, and the ellipticity was measured and recorded at 260 nm, a maximum CD adsorption wavelength of the enantiomerically pure naphthyl-leucine ester, (see, FIG. 1). The data obtained for all the 16 wells are summarized in the Table below.

TABLE
Ellipticities (mdeg) measured at 260 nm for each member of
each parallel L-(4 × 4) library, where the first number represents
data on AmPS resin and the second number represents data on
AmTG resin.

| | AmPS | AmTG |
|---|---|---|
| [Module-1]-[Module-2] | Resin | Resin |
| Anth-Ala | −0.28 | 0.32 |
| Anth-Gly | −0.27 | −0.39 |
| Anth-Leu | 0.28 | 0.20 |
| Anth-Pro | −0.56 | 0.26 |
| Bz-Ala | −0.44 | −0.49 |
| Bz-Gly | 0.12 | −0.04 |
| Bz-Leu | −0.02 | −0.30 |
| Bz-Pro | −0.19 | −0.26 |
| Dnb-Ala | 8.26 | 4.53 |
| Dnb-Gly | −0.06 | −0.18 |
| Dnb-Leu | 13.5 | 7.84 |
| Dnb-Pro | 0.02 | −0.02 |
| Naph-Ala | −0.12 | 0.50 |
| Naph-Gly | −0.10 | −0.11 |
| Naph-Leu | 0.02 | 0.15 |
| Naph-Pro | 0.17 | −0.28 |

As can be seen from the above Table, the measured ellipticities of the internal negative controls (all the Gly data) on AmPS resin ranged from −0.27 to 0.12, and on AmTG resin ranged from −0.39 to −0.04. Since no enantiomeric selectivity is expected from these Gly negative controls, −0.27 to 0.12 and −0.39 to −0.04 should be a good measure of the noise level. The ellipticities of only two wells, namely Dnb-Ala and Dnb-Leu, were well above the noise level, exhibiting significantly large chiral selectivity. Therefore, two potential chiral selectors, Dnb-Ala and Dnb-Leu, were identified from this library.

### Example V

Preparation of immobilized Dnb-Ala-Abu-silica gel stationary phase and immobilized Dnb-Leu-Abu-silica gel stationary phase, and resolution of racemic (1-naphthyl)leucine ester

Both stationary phases were prepared following similar procedures as reported for the preparation of Dnb-Ala-Gly-silica gel by Wu, Wang, Yang, and Li, in “Screening of Mixture Combinatorial Libraries for Chiral Selectors . . . ” noted above. The two potential chiral selectors (Dnb-Ala and Dnb-Leu determined from Example IV above) were then each immobilized onto silica gel (ALLSPHERE) as follows:

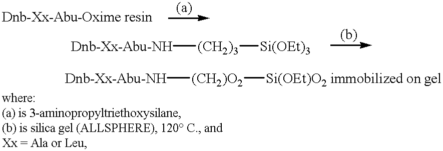

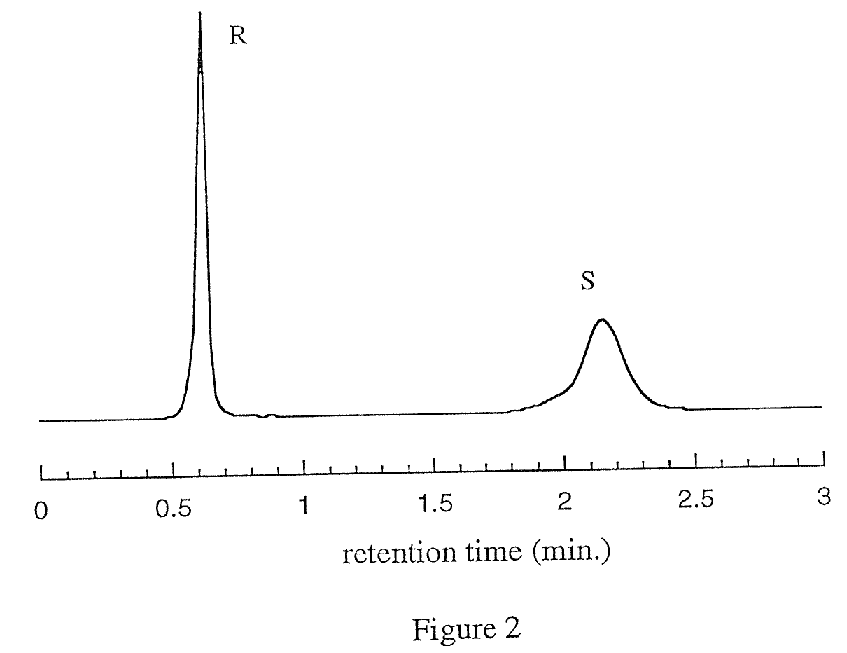

and the resulting stationary phases were individually packed into columns using a standard slurry packing method as reported by Poole and Poole, “Chromatography Today”, Elsevier, pp. 350-353 (1991). (SELECTO silica gel was used to purify Dnb-Xx-Abu-NH-(CH₂)₃-Si(OEt)₃ needed to prepare the 2 columns.) Both silica gel columns were found to work in resolving racemic (1-naphthyl) leucine ester. For instance, the column with L-Leu on silica gel resolved racemic (1-naphthyl)leucine ester well, the retention of the R-enantiomer being about 0.6 minute, while that of the S-being about 2.2 minutes, as can be seen in FIG. 2.

Employing the method according to Pirkle and Welch, Vol. 4, Journal of Liquid Chromatography A, pp. 1-8 (1991), the separation factor α was determined using the following equations:

α=k2/k1

k=(tᵣ-tₒ)/tₒ

where k is the retention factor, tᵣ is the retention time, and tₒ is the dead time determined with 1,3,5-tri-t-butylbenzene as the void volume marker. The separation factor with the Dnb-Ala silica gel column was 4.7, while that of the Dnb-Leu silica gel column was 12.

The ligand surface concentrations were estimated to be 0.24 mmol per gram silica gel for the Dnb-Ala-Abu stationary phase and 0.11 mmol per gram silica gel for the Dnb-Leu-Abu stationary phase, by the nitrogen percentages of both stationary phases determined with elemental analysis.

It will be understood that various details of the invention may be changed without departing from the scope of the invention. Furthermore, the foregoing description is for the purpose of illustration only, and not for the purpose of limitation-the invention being defined by the claims.

What is claimed is:

1. A method for screening chiral selectors from a parallel library comprising:
(a) forming a parallel library by individually synthesizing potential chiral selectors onto a polymeric synthesis resin, where the selectors are chiral, and the selectors are not a mixture of both levo rotational orientation and dextro rotational orientation;
(b) incubating each individual chiral selector, attached on the polymeric resin, with an analyte having a mixture of a R-enantiomer and a S-enantiomer; and
(c) analyzing the resultant of step (b) to identify which chiral selector selectively adsorbed one of the R-enantiomer and the S-enantiomer.

2. The method of claim 1, wherein the polymeric resin in step (a) contains surface amino groups.

3. The method of claim 1, wherein the polymeric resin in step (a) is cross-linked.

4. The method of claim 1, wherein the polymeric resin in step (a) is rigid.

5. The method of claim 1, wherein the polymeric resin in step (a) is selected from the group consisting of polystyrene resin, polyacrylamide resin, and combinations thereof.

6. The method of claim 1, wherein synthesizing in step (a) is accomplished with N-(9-fluorenylmethoxycarbonyl).

7. The method of claim 1, wherein the potential chiral selectors are peptides.

8. The method of claim 1, further including at least one selector that is a negative control.

9. The method of claim 8, where the negative control is selected from the group consisting of a chiral and all racemic.

10. The method of claim 1, wherein the analyte in step (b) is racemic.

11. The method of claim 10, wherein the racemic analyte is (2-naphthyl)leucine ester.

12. The method of claim 1, where incubating in step (b) is accomplished with a batch process.

13. The method of claim 1, wherein analyzing in step (c) is accomplished with circular dichroism.

14. The method of claim 1, wherein the method is free of first immobilizing the analyte in step (b) prior to forming the library in step (a) and analyzing in step (c).

15. The method of claim 1, further including:
(d) attaching the identified chiral selector onto a support; and
(e) resolving the analyte into the R-enantiomer and the S-enantiomer with the attached chiral selector on the support.

16. The method of claim 15, wherein the support is silica gel.

17. The method of claim 15, wherein the analyte is racemic.

18. The method of claim 17, wherein the racemic analyte is (2-naphthyl)leucine ester.
